(19) United States
(12) Patent Application Publication
(10) Pub. No.: US 2022/0395007 A1
(43) Pub. Date: Dec. 15, 2022

(54) TASTE MODULATING COMPOUNDS AND METHODS OF IMPROVING THE QUALITY OF FOODS AND BEVERAGES

(71) Applicant: Ohio State Innovation Foundation, Columbus, OH (US)

(72) Inventors: Devin PETERSON, Upper Arlington, OH (US); Chengyu GAO, Columbus, OH (US)

(21) Appl. No.: 17/770,975

(22) PCT Filed: Oct. 23, 2020
(86) PCT No.: PCT/US20/57180

§ 371 Date: Apr. 21, 2022

Related U.S. Application Data
(60) Provisional application No. 62/924,946, filed on Oct. 23, 2019.
(60) Provisional application No. 63/032,944, filed on Jun. 1, 2020.

(51) Int. Cl. A23L 27/20; C07H 15/24; A23L 27/00; A23F 5/14

(52) U.S. Cl. CPC A23L 27/204; C07H 15/24; A23L 27/86; A23F 5/14; A23V 2002/00

(57) ABSTRACT

Disclosed herein are small molecule compounds that can be used to modulate flavors in foods, beverages, and other articles intended for delivery to the oral cavity. The compounds include terpenoids, cinnamoyl and caffeic esters as disclosed herein. Suitable foods and beverages include coffee, beers, whole grains and vegetables.

### CROSS-REFERENCE TO RELATED APPLICATION

This application claims the benefit of U.S. Provisional Applications 62/924,946, filed on Oct. 23, 2019, and 63/032,944, filed on Jun. 1, 2020, the contents of each are hereby incorporated in their entirety.

### FIELD OF THE INVENTION

The invention relates to flavor modulating compounds and their use as an additive for food and drink. By blocking/modulating bitter flavors, the amount of added sugars and/or salts can be reduced.

### BACKGROUND

There are five primary tastes perceived by the human tongue: salt, sour, sweet, bitter, and umami (i.e., savory). Many people consider the bitter sensation to be unpleasant, and it is speculated that the ability to sense bitterness evolved as an avoidance mechanism against toxic plants and animals. Nevertheless, many foods with high nutritional value, for instance cruciferous vegetables, whole grain foods, and cranberries, also have bitter flavors. These foods are often prepared with high levels of fats, sugars, and/or salts in order to mask the bitterness. Although these additives increase the palatability of the nutritious foods, excess consumption of fat, sugar, and salt is considered unhealthy. As an alternative, bitter blocking compounds having been developed as an additive for foods and vegetables. However, different foods have different distributions of bitter compounds, and many additives only block a subset of bitter flavors.

There remains a need for novel compounds that block the bitter sensations in foods and beverages. The remains a need for compounds that modulate the flavors of foods and beverages. There remains a need for palatable foodstuffs with reduced sugar and salt additives.

### BRIEF DESCRIPTION OF THE FIGURES

FIG. 1 depicts the bitter intensity values for a control coffee, control coffee+4-O-caffeoylquinic acid, control coffee+5-O-caffeoylquinic acid, and control coffee plus 2-O-β-D-glucopyranosyl-atractyligenin.

FIG. 2 depicts the bitter intensity values for a control coffee, control coffee with pH adjustment, and a control coffee with 4-O-caffeoylquinic acid, 5-O-caffeoylquinic acid, and 2-O-β-D-glucopyranosyl-atractyligenin.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The term “alkyl” as used herein is a branched or unbranched hydrocarbon group such as methyl, ethyl, n-propyl, isopropyl, n-butyl, isobutyl, t-butyl, pentyl, hexyl, heptyl, octyl, nonyl, decyl, dodecyl, and the like. The alkyl group can also be substituted or unsubstituted. Unless stated otherwise, the term “alkyl” contemplates both substituted and unsubstituted alkyl groups. The alkyl group can be substituted with one or more groups including, but not limited to, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. An alkyl group which contains no double or triple carbon-carbon bonds is designated a saturated alkyl group, whereas an alkyl group having one or more such bonds is designated an unsaturated alkyl group. Unsaturated alkyl groups having a double bond can be designated alkenyl groups, and unsaturated alkyl groups having a triple bond can be designated alkynyl groups. Unless specified to the contrary, the term alkyl embraces both saturated and unsaturated groups.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is replaced with a heteroatom such as, but not limited to, nitrogen, oxygen, sulfur, selenium or phosphorus. The cycloalkyl group and heterocycloalkyl group can be substituted or unsubstituted. Unless stated otherwise, the terms “cycloalkyl” and “heterocycloalkyl” contemplate both substituted and unsubstituted cyloalkyl and heterocycloalkyl groups. The cycloalkyl group and heterocycloalkyl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. A cycloalkyl group which contains no double or triple carbon-carbon bonds is designated a saturated cycloalkyl group, whereas an cycloalkyl group having one or more such bonds (yet is still not aromatic) is designated an unsaturated cycloalkyl group. Unless specified to the contrary, the term cycloalkyl embraces both saturated and unsaturated, non-aromatic, ring systems.

The term “aryl” as used herein is an aromatic ring composed of carbon atoms. Examples of aryl groups include, but are not limited to, phenyl and naphthyl, etc. The term “heteroaryl” is an aryl group as defined above where at least one of the carbon atoms of the ring is replaced with a heteroatom such as, but not limited to, nitrogen, oxygen, sulfur, selenium or phosphorus. The aryl group and heteroaryl group can be substituted or unsubstituted. Unless stated otherwise, the terms “aryl” and “heteroaryl” contemplate both substituted and unsubstituted aryl and heteroaryl groups. The aryl group and heteroaryl group can be substituted with one or more groups including, but not limited to, alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol.

Exemplary heteroaryl and heterocyclyl rings include: benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyL cirrnolinyl, decahydroquinolinyl, 1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl.

The terms “alkoxy,” “cycloalkoxy,” “heterocycloalkoxy,” “cycloalkoxy,” “aryloxy,” and “heteroaryloxy” have the aforementioned meanings for alkyl, cycloalkyl, heterocycloalkyl, aryl and heteroaryl, further providing said group is connected via an oxygen atom.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Unless specifically stated, a substituent that is said to be “substituted” is meant that the substituent can be substituted with one or more of the following: alkyl, alkoxy, alkenyl, alkynyl, cycloalkyl, heterocycloalkyl, aryl, heteroaryl, aldehyde, amino, carboxylic acid, ester, ether, halide, hydroxy, ketone, nitro, silyl, sulfo-oxo, or thiol. In a specific example, groups that are said to be substituted are substituted with a protic group, which is a group that can be protonated or deprotonated, depending on the pH.

Acceptable salts are salts that retain the desired flavor modulating activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, and all possible geometric isomers.

Disclosed herein are flavor modulating compounds of Formula (1):

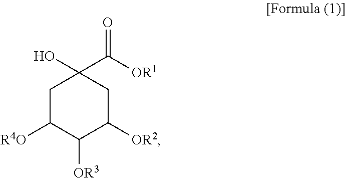

wherein one of R², R³, and R⁴ is a group having the formula:

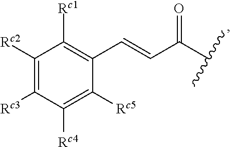

wherein R^{c1}, R^{c2}, R^{c3}, R^{c4}, and R^{c5} are independently selected from H and OH;
the other two of R², R³, and R⁴ are H; and
R¹ is H or C₁₋₈alkyl, wherein said alkyl group may be optionally substituted one or more times.

In certain embodiments, one of R², R³, and R⁴ can form a bond with R′, providing a bicyclic lactone compound.

In preferred embodiments, R^{c1}, R^{c2}, and R^{c5} are each hydrogen, and R^{c2} and R^{c3} are each OH.

The compound of Formula (1) can in some instances be defined when R² is a group having the formula:

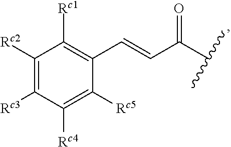

wherein R^{c1}, R^{c2} and R^{c5} are each hydrogen, R^{c2} and R^{c3} are each OH; and each of R¹, R³, and R⁴ are H.

The compound of Formula (1) can in some instances be defined when R³ is a group having the formula:

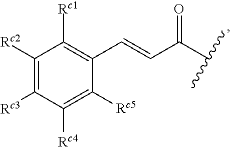

wherein R^{c1}, R^{c4}, and R^{c5} are each hydrogen, R^{c2} and R^{c3} are each OH; and each of R¹, R², and R⁴ are H.

The compound of Formula (1) can in some instances be defined when R⁴ is a group having the formula:

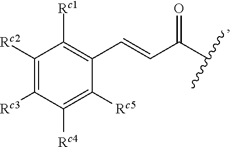

wherein R^{c1}, R^{c4}, and R^{c5} are each hydrogen, R^{c2} and R^{c3} are each OH; and each of R¹, R², and R³ are H.

In some instances, the flavor modulating compound can be a compound of Formula (1a)

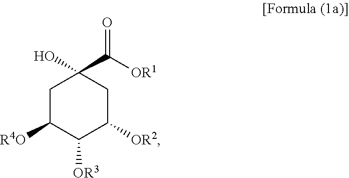

wherein R¹, R², R³, and R⁴ are as defined above.

In some instances, the flavor modulating compound can be a compound of Formula (1b)

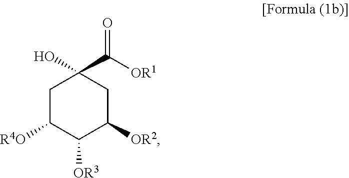

wherein R¹, R², R³, and R⁴ are as defined above.

In some instances, the flavor modulating compound can be a compound of Formula (1c)

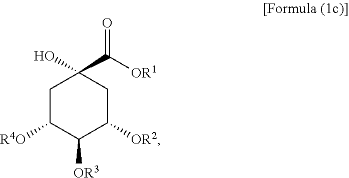

wherein R², R³, and R⁴ are as defined above.
In some instances, the flavor modulating compound can be a compound of Formula (1d)

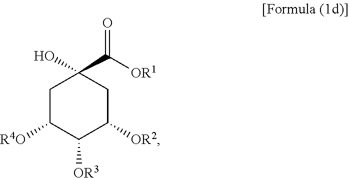

wherein R¹, R², R³, and R⁴ are as defined above.

In some instances, the flavor modulating compound can be a compound of Formula (1e)

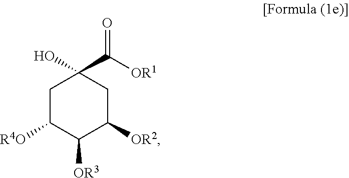

wherein R¹, R², R³, and R⁴ are as defined above.
In some instances, the flavor modulating compound can be a compound of Formula (1f)

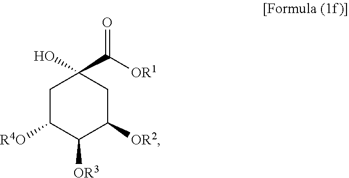

wherein R¹, R², R³, and R⁴ are as defined above.

In some instances, the flavor modulating compound can be a compound of Formula (1g)

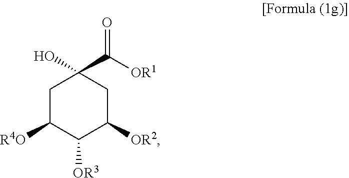

wherein R¹, R², R³, and R⁴ are as defined above.

In some instances, the flavor modulating compound can be a compound of Formula (1h)

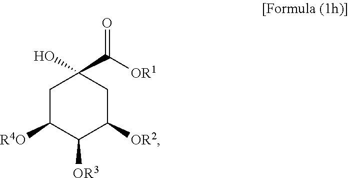

wherein R¹, R², R³, and R⁴ are as defined above.

Preferred flavor modulating compounds 4-O-caffeoylquinic acid having the formula:

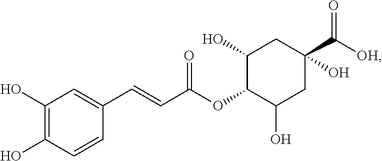 [SMILES: O=C(/C=C/c1ccc(O)c(O)c1)O[C@H]1C(O)C[C@](O)(C(=O)O)C[C@H]1O]

and 5-O-caffeoylquinic acid having the formula:

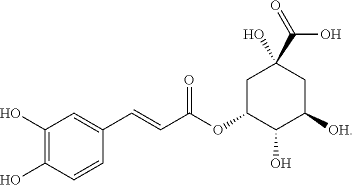 [SMILES: O=C(/C=C/c1ccc(O)c(O)c1)O[C@@H]1C[C@@](O)(C(=O)O)C[C@@H](O)[C@@H]1O]

Also disclosed herein are flavor modulating compounds of Formula (2):

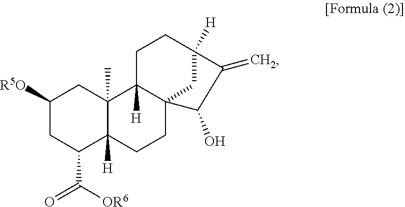

wherein R⁶ is H or C₁₋₈alkyl, wherein said alkyl group may be optionally substituted one or more times; and
R⁵ is H or a carbohydrate residue having the formula:

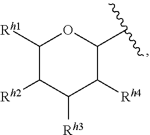

wherein:
R^{h1} is selected from H and CH₂OR^{h1*}, wherein R^{h1*} is selected from H, C₁₋₄alkyl, and C(O)C₁₋₈alkyl;
R^{h2} is selected from H and OR^{h2*}, wherein R^{h2*} is selected from H, C₁₋₄alkyl, and C(O)C₁₋₈alkyl;
R^{h3} is selected from H and OR^{h3*}, wherein R^{h3*} is selected from H, C₁₋₄alkyl, and C(O)C₁₋₈alkyl; and
R^{h4} is selected from H and OR^{h4*}, wherein R^{h4*} is selected from H, C₁₋₄alkyl, and C(O)C₁₋₈alkyl.

In certain embodiments, R^{h1} is CH₂OH, and each of R^{h2}, R^{h3}, and R^{h4} are OH.

In certain embodiments, R⁵ is a carbohydrate residue having the formula

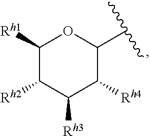

wherein R^{h1}, R^{h2}, R^{h3}, and R^{h4} are as defined above.

In certain embodiments, R⁵ is a carbohydrate residue having the formula

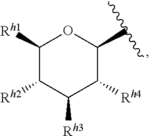

wherein R^{h1}, R^{h2}, R^{h3}, and R^{h4} are as defined above.

Other possible configurations of the carbohydrate residue include:

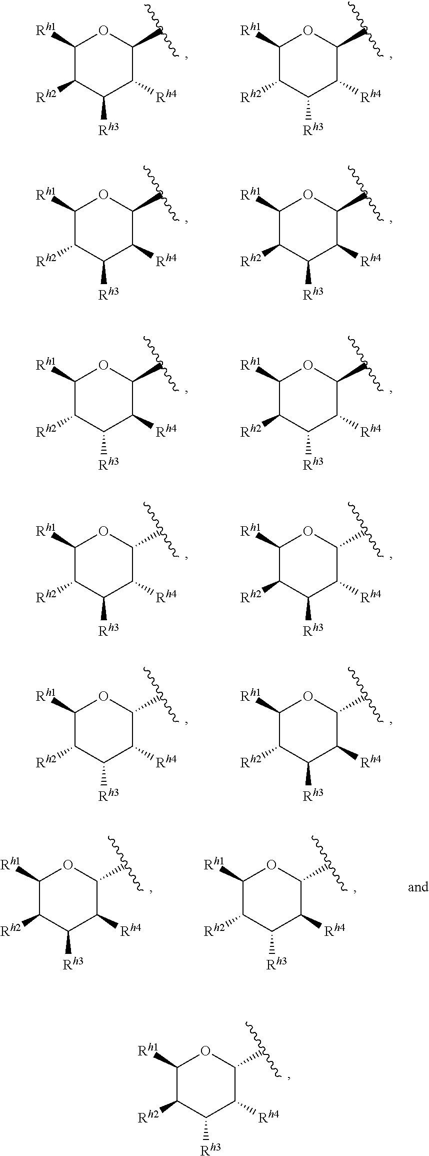

wherein R^{h1}, R^{h2}, R^{h3}, and R^{h4} are as defined above.

The flavor modulating compounds disclosed herein may be obtained by selective esterification of chlorogenic acid, i.e, intramolecular lactonization, esterification of the 4-hydroxyl, followed by lactone hydrolysis. In other embodiments, the flavor modulating compounds may be obtained from coffee beans, preferably green coffee beans, most preferably green coffee beans that are considered below specialty grade. The flavor modulating compounds may be solvent extracted and purified using chromatographic methods.

The flavor modulating compounds may be used to reduce or eliminate the perception of bitter tastes in a variety of foods and beverages. Moreover, the flavor modulating compounds can modulate the aroma and/or somatosensory characteristics of a food or beverage The compounds may be added to a variety of different foods to increase palatability. For instance, the compounds may be added to vegetables, including cruciferous vegetables, yogurts, fruits such as cranberries and other bitter fruits, cocoa, coffee, wine, or beer. In certain embodiments, the flavor modulating compounds can be used to mask the taste of anti-oxidants and preservatives, thereby increasing a food's shelf life without compromising its flavor. The flavor modulating compounds may be added to the wettable adhesives found in stamps and envelopes. The flavor modulating compounds may be added to medications, including liquid formulations, chewable formulations, dissolvable formulations, aerosol formulations, dry powder formulations, and spray formulations. By reducing bitterness, an increased adherence to a treatment regimen can be achieved, especially with pediatric patients. In other embodiments, the flavor modulating compounds may be combined with dental formulations, including topical anesthetics, adhesives, including denture adhesives, and cleaning products such as toothpastes, mouthwashes, and sealants.

The flavor modulating compounds may be added to foods and beverages in concentrations effective to block the bitter tastes of the compounds contained therein. The compounds can augment or improve flavors as well as somatosensory effects (e.g., warming cooling sensations) in foods and beverages. The flavor modulating compounds can improve and/or augment the aromas associated with a particular food or beverage. For instance, the disclosed compounds may be added in an amount of at least 0.1 mg/kg, at least 0.5 mg/kg, at least 1 mg/kg, at least 2.5 mg/kg, at least 5 mg/kg, at least 10 mg/kg, at least 20 mg/kg, at least 30 mg/kg, at least 40 mg/kg, at least 50 mg/kg, at least 60 mg/kg, at least 70 mg/kg, at least 80 mg/kg, at least 90 mg/kg, at least 100 mg/kg, at least 200 mg/kg, at least 250 mg/kg, at least 500 mg/kg, at least 750 mg/kg, or at least 1,000 mg/kg, relative to the total weight of the consumable. In some embodiments, the flavor modulating compound can be added in an amount from 0.1-1,000 mg/kg, from 0.1-750 mg/kg, from 0.1-500 mg/kg, from 0.1-250 mg/kg, from 0.1-100 mg/kg, from 0.1-50 mg/kg, from 0.1-25 mg/kg, from 0.1-10 mg/kg, from 0.1-5 mg/kg, from 0.1-2.5 mg/kg, from 5-100 mg/kg, from 5-50 mg/kg, from 5-25 mg/kg, from 5-10 mg/kg, from 10-100 mg/kg, from 10-50 mg/kg, from 10-25 mg/kg, from 25-100 mg/kg, from 25-50 mg/kg, from 50-100 mg/kg, from 75-100 mg/kg, from 25-250 mg/kg, from 50-250 mg/kg, from 75-250 mg/kg, or from 100-250 mg/kg.

In certain embodiments, the flavor modulating compounds can be delivered to the oral cavity prior to consumption of the bitter product. The compounds can be formulated as a mouthwash, a lozenge, a lollipop, a chewable tablet, and the like. By pre-saturating the bitter taste receptors in the tongue with the flavor modulating compounds, otherwise unpalatable substances may be more readily delivered to the oral cavity or consumed.

The compounds disclosed herein may be provided in an aqueous composition to more readily combine them with foods, beverages, and the like. The composition may be buffered, for instance at a pH between 6 and 8, between 6.5 and 8, between 6.5 and 7, between 6.5 and 7.5, between 6.5 and 8, between 7 and 8, or between 7.5 and 8. In other embodiments, the composition may be buffered at an acidic pH, for instance similar to found in citrus juice, vinegar, or yogurt. In some embodiment, the composition may be buffered at a pH between 2 and 8, between 2 and 7, between 2 and 6, between 2 and 5, between 2 and 4, between 3 and 8, between 3 and 7, between 3 and 6, between 3 and 5, between 3 and 4, between 4 and 8, between 4 and 7, between 4 and 6, between 4 and 5, between 5 and 8, between 5 and 7, or between 5 and 6.

The compounds may be provided in the composition at a concentration between about 0.1-100 mM, between about 0.5-100 mM, between about 1-100 mM, between about 5-100 mM, between about 10-100 mM, between about 25-100 mM, between about 50-100 mM, between about 0.1-50 mM, between about 0.1-25 mM, between about 0.1-10 mM, between about 0.1-5 mM, or between about 0.1-1 mM. When the aqueous composition contains more than one flavor modulating compound, the concentration refers to the total concentration of all the compounds.

In some embodiments, at least one compound of Formula 1 can be combined with at least one compound of Formula 2 to further modify flavor as described above. In a particular embodiment, one of 4-O-caffeoylquinic acid and 5-O-caffeoylquinic acid (or both) is combined with 2-O-β-D-glucopyranosyl-atractyligenin having the structure:

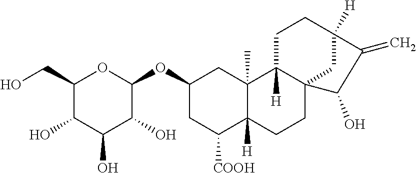

### Example

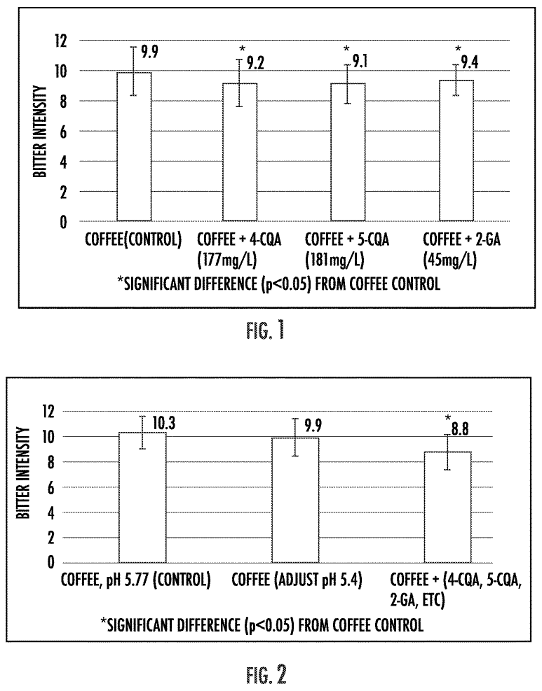

4-CQA (4-caffeoylquinic acid) (about 177 mg/L), 5-CQA (5-caffeoylquinic acid) (about 181 mg/L), and 2-GA (2-O-β-D-glucopyranosyl-atractyligenin) (about 45 mg/L) were added individually or as a group to a coffee rated as having a high bitter intensity. Five mL of each sample at room temperature was presented to experienced sensory panelists in 1 oz black cups labeled with 3-digit codes in random order. Eight trained panelists, recruited by Flavor Research and Education Center (FREC) at The Ohio State University, used nose clips during the evaluation in order to prevent interactions with olfactory responses. Between samples, panelists waited a minimum of 90 seconds and were asked to cleanse their palate using crackers and water. All evaluations were conducted in duplicate using Compusense Cloud software (Compusense Inc., Guelph, ON, Canada). The addition of 4-CQA, 5-CQA or 2-GA individually or as a group were reported to significantly reduce (p<0.05) the perceived bitterness intensity of the coffee brew. FIGS. 1 and 2.

The addition of the compounds reduced the sample pH from 5.77 to 5.40, therefore, to evaluate the effect of pH, an additional control sample with an adjusted pH of 5.40 was also evaluated. No significant difference in bitterness intensity was noted for this change in coffee pH compared to the control, confirming the reduction of coffee bitterness was impacted by the addition of negative markers and not the corresponding change of pH value. FIG. 2.

The taste attributes 4-CQA, 5-CQA, and 2-GA in isolation were further evaluated by 5 experienced sensory panelists by consensus. Each compound was dissolved in 5 mL nanopure water and 5 mL buffer (0.05 M sodium phosphate butter adjusted to pH 5.77 with 0.1 M citric acid) at the same concentrations described above. A very weak sour taste was reported for 4-CQA and 5-CQA in both water and buffer, while no obvious taste character was observed for 2-O-β-D-glucopyranosyl-atractyligenin in either water or the buffer solution. This led to the confirmation that 4-CQA, 5-CQA, and 2-O-β-D-glucopyranosyl-atractyligenin were bitter modulators that suppressed perception.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A method for reducing bitterness is a food product, comprising adding to the food product:
a flavor modifying compound of Formula (1): 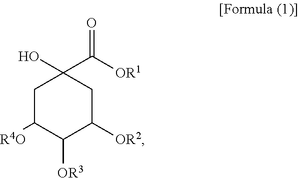
or an acceptable salt thereof,
wherein one of R², R³, and R⁴ is a group having the formula: 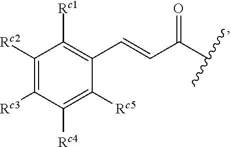
wherein R^{c1}, R^{c2}, R^{c3}, R^{c4} and R^{c5} are independently selected from H and OH;
the other two of R², R³, and R⁴ are H; and
R¹ is H or C₁₋₈alkyl;
a flavor modifying compound of Formula (2): 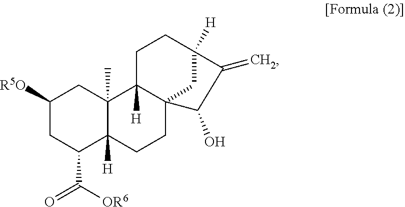
wherein R⁶ is H or C₁₋₈alkyl, and
R⁵ is H or a carbohydrate residue having the formula: 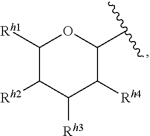
wherein:
R^{h1} is selected from H and CH₂OR^{h1*}, wherein R^{h1*} is selected from H, C₁₋₄alkyl, and C(O)C₁₋₈alkyl;
R^{h2} is selected from H and OR^{h2*}, wherein R^{h2*} is selected from H, C₁₋₄alkyl, and C(O)C₁₋₈alkyl;
R^{h3} is selected from H and OR^{h3*}, wherein R^{h3*} is selected from H, C₁₋₄alkyl, and C(O)C₁₋₈alkyl; and
R^{h4} is selected from H and OR^{h4*}, wherein R^{h4*} is selected from H, C₁₋₄alkyl, and C(O)C₁₋₈alkyl;
or a combination of a compound of formula (1) and formula (2).

2. The method according to claim 1, wherein R^{c1}, R^{c4}, and R^{c5} are each hydrogen, R^{c2} and R^{c3} are each OH; and each of R¹, R³, and R⁴ are H.

3. The method according to claim 1, R² is a group having the formula: 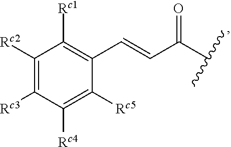
wherein R^{c1}, R^{c4}, and R^{c5} are each hydrogen, R^{c2} and R^{c3} are each OH; and each of R¹, R³, and R⁴ are H.

4. The method according to claim 1, wherein R³ is a group having the formula: 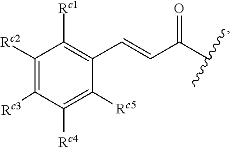
wherein R^{c1}, R^{c4}, and R^{c5} are each hydrogen, R^{c2} and R^{c3} are each OH; and each of R¹, R², and R⁴ are H.

5. The method according to claim 1, wherein R⁴ is a group having the formula: 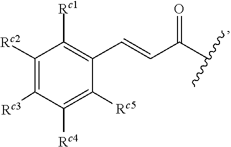
wherein R^{c1}, R^{c4}, and R^{c5} are each hydrogen, R^{c2} and R^{c3} are each OH; and each of R¹, R², and R³ are H.

6. The method according to claim 1, wherein the compound of Formula (I) has the structure:

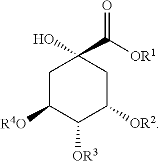

7. The method according to claim 1, wherein the compound of Formula (I) has the structure:

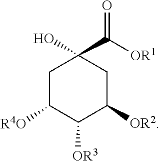

8. The method according to claim 1, wherein the compound of Formula (I) has the structure:

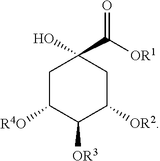

9. The method according to claim 1, wherein the compound of Formula (I) has the structure:

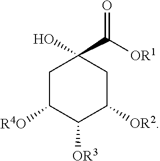

10. The method according to claim 1, wherein the compound of Formula (I) has the structure:

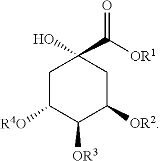

11. The method according to claim 1, wherein the compound of Formula (I) has the structure:

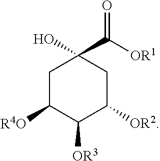

12. The method according to claim 1, wherein the compound of Formula (I) has the structure:

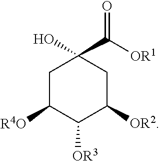

13. The method according to claim 1, wherein the compound of Formula (I) has the structure:

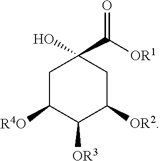

14. The method according to claim 1, wherein R⁵ is a carbohydrate residue having the formula

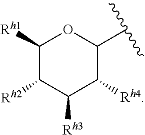

15. The method according to claim 1, wherein R⁵ is a carbohydrate residue having the formula

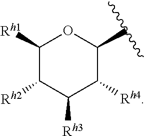

16. The method according to claim 1, wherein R⁵ is a carbohydrate residue having the formula

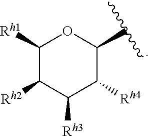

17. The method according to claim 1, wherein R⁵ is a carbohydrate residue having the formula

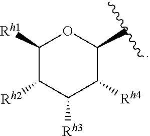

18. The method according to claim 1, wherein R⁵ is a carbohydrate residue having the formula

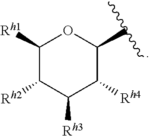

19. The method according to claim 1, wherein R^{h1} is CH₂OH, R^{h2} is OH, wherein R^{h3} is OH, and R^{h4} is H, OH, NH₂, NH(CO)CH₃.

20-25. (canceled)

26. The method of claim 1, wherein the food product comprises a coffee, a vegetable, a yogurt, a whole grain, a beer, a wine, a distilled spirit, a cocoa, a yogurt, or a fruit.
